(19) United States
(12) Patent Application Publication
(10) Pub. No.: US 2025/0033037 A1
(43) Pub. Date: Jan. 30, 2025

(54) MICROCHANNEL SPERM CELL PREPARATION

(71) Applicant: THE UNIVERSITY OF BIRMINGHAM, Birmingham (GB)

(72) Inventors: Jackson Kirkman-Brown, Birmingham (GB); Meurig Thomas Gallagher, Birmingham (GB); David Smith, Birmingham (GB)

(21) Appl. No.: 18/709,455

(22) PCT Filed: Nov. 11, 2022
(86) PCT No.: PCT/GB2022/052873

§ 371 Date: May 10, 2024

(30) Foreign Application Priority Data
Nov. 12, 2021 (GB) 2116389.4

(51) Int. Cl. B01L 3/00; C12N 5/071

(52) U.S. Cl. CPC B01L 3/5027; C12N 5/0612; B01L 2200/0652; B01L 2300/0816; B01L 2300/0861

(57) ABSTRACT

The invention relates to a microchannel device for preparation of sperm, the device comprising: an inlet; an extraction zone; and a channel fluidly connecting the inlet to the extraction zone; wherein the channel comprises a plurality of orienting features operable to bias a direction of sperm cell movement within the channel towards a preferred direction; and wherein the channel has a length of approximately 1 centimetre or greater. Further, the invention relates to a method of sperm preparation using the microchannel device.

### TECHNICAL FIELD

The present invention relates to microchannel devices and methods for sperm cell preparation. The devices and methods are particularly, but not exclusively, useful in assistive reproductive technologies such as intrauterine insemination (IUI) in vitro fertilisation (IVF) and intracytoplasmic sperm injection (ICSI).

### BACKGROUND

It is reported that one in six couples experience fertility problems. Fertility treatments in the form of assisted reproductive technologies (ARTs) can be used to assist those experiencing fertility problems. The majority of ARTs involve external handling of oocytes, spermatozoa (herein “sperm”), and embryos, with the aim of establishing a pregnancy, and ultimately a live birth.

The two main forms of ART are in vitro fertilisation (IVF) and intracytoplasmic sperm injection (ICSI). IVF involves approximately 500,000 sperm cells being exposed to an oocyte, whereas individual sperm cells are directly injected into the oocyte cytoplasm in ICSI. The resulting embryo from either method is introduced into the uterus after two to five days. Other forms of ART include intrauterine insemination (IUI), where prepared samples usually containing over 3 million sperm cells are injected into the uterus.

ARTs are becoming increasingly frequent procedures, with the Human Fertilisation and Embryology Authority (HFEA) seeing annual patients rise from 600,000 to 800,000 between 2010 and 2016. However, ART success rates remain poor at around 30% per cycle. Not only can failures negatively impact patient physical and mental health, but they are also an inefficient usage of healthcare resources because many patients will attempt further cycles in pursuit of a child despite not being guaranteed success. Improving ART success would therefore be beneficial for both patients and healthcare resources. ART techniques are equally applicable in other animal species for breeding, conservation and toxicology.

It is theorised that there exists a strong in vivo sperm selection process in natural conception based on multiple quality parameters, such that only around 100 sperm cells reach the site of fertilisation from an ejaculate containing approximately 250 million cells. It has been hypothesised that this natural selection process means that only high-quality spermatozoa are able to fertilise the oocyte, thereby maximising the chances of live birth.

Sperm preparation is central in the ART process for removal of microbes and fertilisation inhibitors. It also aims to isolate a healthy cell population from a sample containing many abnormal cells.

Current ART sperm preparation methods generally weakly select using a singular indicator of sperm quality, so low-quality sperm are likely to be employed than in in vivo selection, potentially contributing to the high rate of ART failures. Sperm quality can vary in several ways, for example in cell quantity, morphology, motility and viability. These parameters will vary between patients, often enabling identification of male infertility, and because there are millions of sperm in each ejaculate, they will also vary between cells from the same sample. Therefore, selection of the highest quality sperm cells from any given ART sample can increase the chance of a high quality sperm fertilising the oocyte, and thereby help maximise the success of treatment.

Density gradient centrifugation (DGC) is a sperm preparation method that can yield useful cell numbers. DGC separates sperm cells according to density, which varies with morphology. Cells with normal morphology are more dense, so form a pellet following centrifugation, whilst spermatozoa with abnormal morphologies can be de-selected on account of their relatively low density. All cells in the sample are sorted by centrifugation, and the density gradient can be altered according to the required balance of cell quality and quantity, so DGC can provide enough cells for use in most ICSI, IVF and IUI procedures. DGC is thus one of the most commonly used sperm preparation methods at the time of writing.

There is however conflicting evidence regarding whether or not DGC introduces DNA damage to the cells. Even if direct damage cannot be conclusively attributed to DGC, centrifugation does increase oxidative stress in sperm cells, which can in turn compromise sperm DNA integrity. The effect of DGC on sperm DNA is important because recent evidence has linked ART failures to DNA quality, a previously ignored sperm quality parameter.

Also in use is an older “swim up” method wherein the sperm cells swim from semen into an overlayered media, but this is prone to contamination with semen and difficult to standardise.

Several sperm selection methods capable of producing high sperm yields are currently being marketed, including micro-electrophoresis, magnetic activated cell sorting (MACS), and microchannels.

Micro-electrophoresis is selection based on the membrane charge of sperm. A more negative charge is associated with increased maturity due to the addition of sialic acids late in spermatogenesis, and importantly, reduced levels of DNA fragmentation.

MACS is focussed on separating cells via deselection of those with surface externalisation of annexin V due to apoptosis, but the method involves centrifugation for seminal plasma removal, so it may introduce cell damage, thereby limiting its effectiveness.

Another mainstay in cell sorting techniques is via antigen-antibody interactions though this may preclude the further use of enriched populations for further use in ART.

Microfluidics involves the use of fluids in systems on a scale of microlitres in channels of predetermined architecture, designed to manipulate assays on account of surface and flow interactions dominating bulk properties. The system design can be manipulated in many ways, so specific, bespoke systems can be made to examine a plethora of structures. Several human systems have been successfully mirrored in this way, such as the liver for models of hepatitis B infection, the heart for modelling heart disease, and the kidney for drug screening.

Microchannel sperm selection to date has involved injecting cells into a shallow chamber and either subjecting them to flow or allowing them to move under their own motility. Cells capable of a degree of active motility are then extracted, giving an enriched population of functional, high quality cells.

Sperm sorting techniques which do not impair sperm quality parameters, and that identify sperm with low DNA fragmentation, have potential to improve ART success rates.

We present herein a device and method for preparation of self-motile sperm cells using microchannels.

### SUMMARY OF THE INVENTION

According to a first aspect of the invention we provide a microchannel device for preparation of sperm, the device comprising:
an inlet;
an extraction zone; and
a channel fluidly connecting the inlet to the extraction zone;
wherein the channel comprises a plurality of orienting features operable to bias a direction of sperm cell movement within the channel towards a preferred direction; and
wherein the channel has a length of approximately 1 centimetre or greater.

On introduction to a device of the type described above, sperm cells are encouraged to swim in the preferred direction which may be towards the extraction zone, by the orienting features. The length of the channel is selected so as to permit motile cells to separate from less motile cells as they swim in the preferred direction. Together these features improve the likelihood of higher quality (i.e. more motile) sperm reaching the extraction zone prior to lower quality sperm within a defined time period (e.g. at least 15 minutes, for example 30-60 minutes). Such a device is thus operable to sort high quality sperm from lower quality sperm without centrifugation.

The channel may comprise a first side wall, a second side wall, a top wall and a bottom wall, together defining a channel volume. The plurality of orienting features may be located in the channel so as to increase the surface area to volume ratio of the channel as compared with a channel absent said orienting features.

Sperm cells are known to preferentially swim along surfaces. Thus, increasing the surface area to volume ratio of the channel increases the likelihood of interactions between sperm cells and the surfaces of the channel and/or orienting features.

The orienting features may comprise one or more internal walls disposed within the channel so as to define a plurality of sub-channels. The device may comprise walls placed throughout its full width, for example more than 4 internal walls, more than 5 internal walls, more than 6 internal walls, between 4 and 20 internal walls, or between 7 and 10 internal walls. The walls may be equally spaced. Together with the side, top and bottom surfaces of the channel, such walls provide a continuous bias to the movement of sperm cells as those cells progress along the channel.

The microchannel device thus may comprise a plurality of sub-channels or compartments which are approximately parallel to one another. Dividing a volume into a plurality of compartments results in a huge surface area for cells to migrate over (as compared with the surface area of the undivided volume), as cells preferentially migrate on a surface not in the volume of liquid held within the compartments.

Each sub-channel may have a width that is small when compared to the full width of the channel (e.g. less than a quarter of the full channel width). The width of each sub-channel may be wide enough to permit a sperm cell to progress through the channel, and preferably wide enough to permit two sperm cells to progress through the channel (one on each wall). Each sub-channel may have a width that is of the same order of magnitude as the length of a sperm cell, in order to be narrow enough to prevent a sperm cell from easily turning. Each channel may have a width that is between 10 μm and 200 μm.

A first end of at least one of the one or more internal walls may be shaped to encourage sperm cell progression into a sub-channel defined at least in part by that internal wall. The first end of each of the one or more internal walls may be rounded or tapered, said first ends being located closest to the inlet.

A second end of at least one of the one or more internal walls may be shaped to discourage sperm cell progression into a sub-channel defined at least in part by that internal wall. The second end of each of the one or more internal walls may comprise a flat surface at an angle to (for example, perpendicular to) the preferred direction, said second ends being located closest to the extraction zone.

The extraction zone may comprise one or more non-return features. The extraction zone may comprise an entry channel, and the non-return features may comprise one or more surfaces shaped to deflect cells away from the entry channel. The surfaces may be curved.

The microchannel device may further comprise a cell transit medium within the device. Alternatively, a cell transit medium may be loaded into the device prior to loading the sample that is to be sorted.

The channel and orienting features may be formed of polydimethylsiloxane (PDMS), polypropylene, polyethelene, polyethelyne terephthalate (PET), biaxially-oriented polypropylene (BOPP), ethylene-vinyl alcohol (EVOH), or other plastic including but not limited to nylon, polyetheretherketone (PEEK), polybutylene terephthalate (PBT), acrylic, acrylonitrile butadiene styrene (ABS), polycarbonate, polysulfone, polystyrene, polyethersulfone (PES), polyphenylsulfone (PPSU), polyvinyl chloride (PVC), or non-plastic material. Similarly, the inlet and extraction zone may be formed of PDMS, including non-return features, if present. Any other suitable material may be used however.

The microchannel device may comprise one or more monitoring regions. A monitoring region may comprise a portion of the device allowing for examination of cells transiting through the device, for instance via microscope. A monitoring region may comprise a transparent or translucent portion of the device. In some cases, one or more walls (e.g. top and/or bottom walls) of the device may be transparent/translucent such that cells may be monitored at any location within the device.

According to a second aspect of the invention, we provide a microchannel device for preparation of sperm, the device comprising:
an inlet;
an extraction zone; and
a plurality of sub-channels fluidly connecting the inlet to the extraction zone;
wherein each sub-channel has a length of approximately 1 centimetre or greater.

As discussed above, each subchannel may have a width of between 10 μm and 200 μm.

A first end of at least one of the sub-channels may comprise an entry feature shaped to encourage sperm cell progression into the sub-channel.

A second end of at least one of the sub-channels may comprise an exit feature shaped to discourage sperm cell progression into the sub-channel.

The sub-channels may be defined by walls disposed within a manifold, such as internal walls located within a channel as discussed above in connection with the first aspect of the invention.

The extraction zone may comprise one or more non-return features. The extraction zone may comprise an entry channel, and the non-return features may comprise one or more surfaces shaped to deflect cells away from the entry channel. The surfaces may be curved.

The microchannel device may further comprise a cell transit medium within the device. Alternatively, a cell transit medium may be loaded into the device prior to loading the sample that is to be sorted.

The microchannel device may not comprise a chemoattractant and/or a chemical concentration gradient intended to select sperm, instead it relies on the walls to allow sperm to navigate.

The one or more components of the microchannel device may be formed of polydimethylsiloxane (PDMS), or other suitable material as discussed above, which in turn my be bonded to another substrate such as glass or plastic material.

According to a third aspect of the invention, we provide a method of sperm preparation, the method comprising:
loading a sample comprising sperm cells into an inlet of a microchannel device as set out in the first aspect of the invention or the second aspect of the invention;
allowing sperm cells to transit towards the extraction zone; and
extracting sperm cells from the extraction zone of the microchannel device.

The method may comprise allowing sperm cells to transit towards the extraction zone for an incubation time sufficient for cells to reach the extraction zone given the length of the channel and the typical speed of motile sperm cells. Such an incubation time may be at least 15 minutes, at least 30 minutes, at least 45 minutes, or at least 60 minutes.

The incubation time may be selected to minimise the likelihood of cells turning at the extraction zone and swimming back towards the inlet, and may thus be less than 2 hours, less than 90 minutes or less than 75 minutes. The method may comprise allowing sperm cells to transit towards the extraction zone for between 15 and 75 minutes, for approximately 30 minutes or for approximately 60 minutes.

The method and organisation of the plurality of channels in the device minimises the likelihood of agitation of semen and other media, such that optimal extraction of non-contaminated media is possible.

Features of each aspect of the invention may be combined with features from other aspects of the invention if required, as well as with features taken from the description which follows below.

### BRIEF DESCRIPTION OF THE DRAWINGS

Reference will now be made, by way of example only, to the accompanying drawings, in which:

FIG. 1 schematically shows a first example of a microchannel device (picture A) together with a control device (picture B);

FIG. 2 illustrates a cross-section through a microchannel device parallel to a longitudinal axis of said device;

FIG. 3 shows experimental versions of the devices schematically shown in FIG. 1;

FIG. 4 illustrates the principle of sperm dispersion in a microchannel device;

FIG. 5 schematically shows a second example of a microchannel device (picture A) together with a control device (picture B);

FIG. 6 illustrates a cross-section through a sub-channel of a microchannel device perpendicular to a longitudinal axis of said device;

FIG. 7 shows the proportion of cells meeting walls in the devices shown in FIG. 1 plotted against the proportion of cells swimming forwards;

FIG. 8 shows the proportion of cells meeting walls in the devices shown in FIG. 5 plotted against the proportion of cells swimming forwards;

FIG. 9 shows average cell concentrations at 1 cm intervals along the devices shown in FIG. 1 30 minutes after loading a sample;

FIG. 10 shows average cell concentrations at 1 cm intervals along the devices shown in FIG. 5 30 minutes after loading a sample;

FIG. 11 shows average cell concentrations at 1 cm intervals along the devices shown in FIG. 1 60 minutes after loading a sample; and

FIG. 12 shows average cell concentrations at 1 cm intervals along the devices shown in FIG. 5 60 minutes after loading a sample,

### DETAILED DESCRIPTION

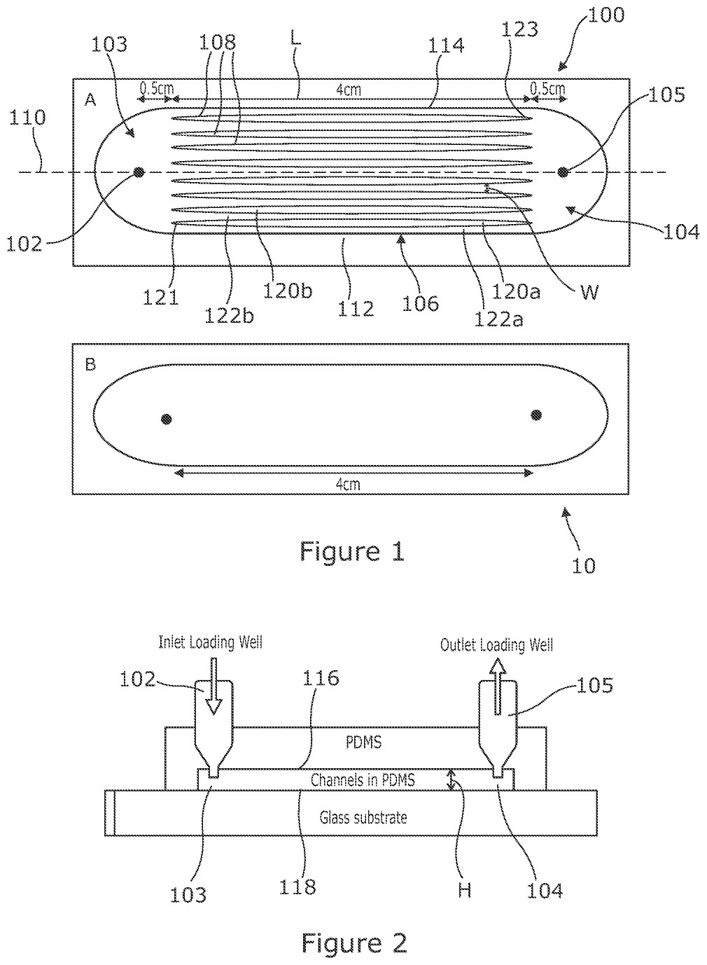

Referring first to FIGS. 1 and 2, a microchannel device 100 for preparation of sperm cells is shown in picture A of FIG. 1. The microchannel device 100 includes one or more inlets 102, an extraction zone 104 and a channel 106 fluidly connecting the inlet to the extraction zone. The inlet permits a liquid substance including sperm cells to be prepared, for example semen, to be introduced to the device 100. The inlet 102 is, in the example shown, located within an inlet zone 103 which fluidly connects the inlet to the channel 106. The extraction zone 104 includes one or more outlets 105, which permit cells that have progressed through the channel to be extracted from the device.

The channel 106 comprises a plurality of orienting features 108 that are operable to bias a direction of sperm cell movement within the channel towards a preferred direction, and in particular to bias the direction of cell movement in a direction from the inlet 102 towards the extraction zone 104. The preferred direction is, in the example shown, parallel to a longitudinal axis 110 of the microchannel device 100.

The channel 106 comprises a first side wall 112, a second side wall 114, a top wall 116 and a bottom wall 118, together defining a channel volume. The plurality of orienting features 108 are located in the channel 106 so as to increase the surface area to volume ratio of the channel as compared with a channel absent said orienting features. An example of a microchannel device including a channel absent orienting features is shown in picture B of FIG. 1 for comparison.

In the example shown, the orienting features comprise one or more internal walls 120 disposed within the channel. The walls 120 define a plurality of sub-channels 122. For example, a first sub-channel 122a is defined between the first channel wall 112 and a first internal wall 120a, a second sub-channel 122b is defined between the first internal wall 120a and a second internal wall 120b, and so on. In a channel 106 including N walls, N+1 sub-channels are defined.

The microchannel device may thus be thought of as a device comprising an inlet 102, an extraction zone 104, and a plurality of sub-channels 122 fluidly connecting the inlet to the extraction zone. Thus subchannels need not be provided via internal walls 120, as shown in FIG. 1. For instance, they could be provided by a plurality of bores such as capillaries. Each sub-channel is defined by one or more channel walls.

Turning back to the example shown in FIG. 1, the device has a plurality of internal walls. In this instance, “a plurality” means more than four internal walls, for example more than 5, 6, 7, 8, 9 or 10 internal walls. The device preferably includes walls placed throughout its full width. The device shown in FIG. 1 includes eight internal walls, thus defining nine sub-channels 122 within the major channel 106.

Evidence suggests that sperm cells are hydrodynamically attracted to, and subsequently swim along surfaces that they meet, which likely reflects their interaction with female tract epithelia. The devices described herein take advantage of this observation to promote the separation of rapidly motile sperm from less motile sperm. Specifically, the devices described herein provide an increased surface area making it more likely that sperm cells will contact an internal surface of the device. Shaping those surfaces in a manner which orients the cells towards the extraction zone encourages those cells to swim in the direction of the extraction zone.

Mathematical dispersion models may explain the effect of surface area on cell motility. Two types of dispersion are the models of random walk, and random walk with persistence. The random walk dispersion model is based on diffusive processes where cell movement is random, unbiased, and not influenced by any previous movement. The chance of a cell being at a given point reduces as the distance from the start site increases, so a graph of expected cell counts along a chip would form a parabola on a log-linear scale. It is believed that this model explains the motion of sperm cells in the absence of any interaction with external objects.

Random walk with persistence is similar, but there is a bias in cell movement which therefore causes persistence in cell movement directions. There is still a reduction in the chance that a cell is reaches a point further from the start site, but the persistence means that the graph of the counts would take a more linear pattern on a log-linear scale. As sperm cells swim along the surfaces they encounter, it is known that surfaces introduce a bias to cell movement, resulting in increased cell progression according to the random walk with persistence model compared to environments with fewer available surfaces where movement is unbiased.

Therefore, the devices described herein provide a plurality of orienting features to increase surface area within a channel so as to provide additional walls for sperm cells to interact with within the channel. The walls are aligned so as to encourage cell motion from the inlet zone towards the extraction zone. In the specific examples shown, the walls are aligned with the longitudinal axis of the channel 106, and are substantially parallel with one another. Such devices reduce random walk behaviour and so improve sperm yields as compared to microchips without additional internal walls.

The walls are substantially free of features which might undesirably bias sperm cell movement, for instance features which might prompt the cells to change direction away from the extraction zone, such as bumps or protrusions. The walls shown have a smooth surface.

The length L of the channel 106, and more particularly the length of the internal walls 120 defining sub-channels 122, is selected to permit separation of rapidly motile sperm (which is more likely to be high quality) from less motile sperm (which is likely to be of lower quality). The length is therefore selected to be a distance which, in a given time period, a highly motile cell might be able to swim but a less motile cell would be less likely to swim. Suitable lengths may be greater than 1 centimetre, greater than 1.5 centimetres, greater than 2 centimetres, greater than 2.5 centimetres, or greater than 3 centimetres. In particular, the channel length may be between 1-6 centimetres, between 1-5 centimetres, between 1-4 centimetres, or between 1.5-4 centimetres. The channel 106 shown in FIG. 1 has a length L of approximately 4 centimetres, not including the inlet zone (which has a length of 0.5 centimetres) or the extraction zone (which also has a length of 0.5 centimetres). The device shown in FIG. 1 is symmetrical, and thus the inlet zone and extraction zone may be reversed if required.

To further encourage cell interaction with the walls of the device each sub-channel has a width W (i.e. a distance between the internal walls) that is narrow when compared with the channel length L. The sub-channel width is selected to maximise the surface area available to sperm cells. The minimum sub-channel width, to allow a sperm cell to swim on each of the available internal walls, should therefore be 10 μm. The maximum sub-channel width should be on the same order of magnitude a sperm cell length, and may be, for example, less than 500 μm, less than 400 μm, between 300-50 μm, between 250-150 μm or less than 200 μm. The sub-channel width W of the device in FIG. 1 is approximately 200 μm.

In addition to a width W defined by the internal walls (and, for the edge-channels, by either the first or second side wall of the main channel), each sub channel may also have a top wall and a bottom wall defined by the top wall 116 and bottom wall 118 of the channel 106. These top and bottom walls define a channel height H. The channel height may be of the same order of magnitude as the channel width W, described above. FIG. 6 shows a schematic view of a cross section through a sub-channel 122, and illustrates that a sperm cell may interact with a top wall of said channel (cell B) as well as or instead of a side wall of said channel (cell A). Similarly, a sperm cell may interact with a bottom wall of said channel as well as or instead of a side wall or top wall of said channel

The principle of using microchannels for sperm sorting is illustrated in FIG. 4. The method comprises a first step a) of loading a sample comprising sperm cells (e.g. a semen sample) into an inlet of a microchannel device, such as the device shown in picture A of FIG. 1. The method comprises a second step b) of allowing sperm cells to transit towards the extraction zone for an incubation time. Finally, a third step c) comprises extracting sperm cells from an extraction zone of the microchannel device.

The method and organisation of the plurality of channels in the device minimises the likelihood of interaction between semen and other media, such that optimal extraction of non-contaminated media is possible.

To allow motile cells to separate from less motile cells the incubation time may be at least 15 minutes, at least 30 minutes, at least 45 minutes, or at least 60 minutes. In particular, the incubation time may be between 15 and 75 minutes, for example approximately 30 minutes, approximately 45 minutes or approximately 60 minutes.

### First-Generation Microchannels Microchip Designs

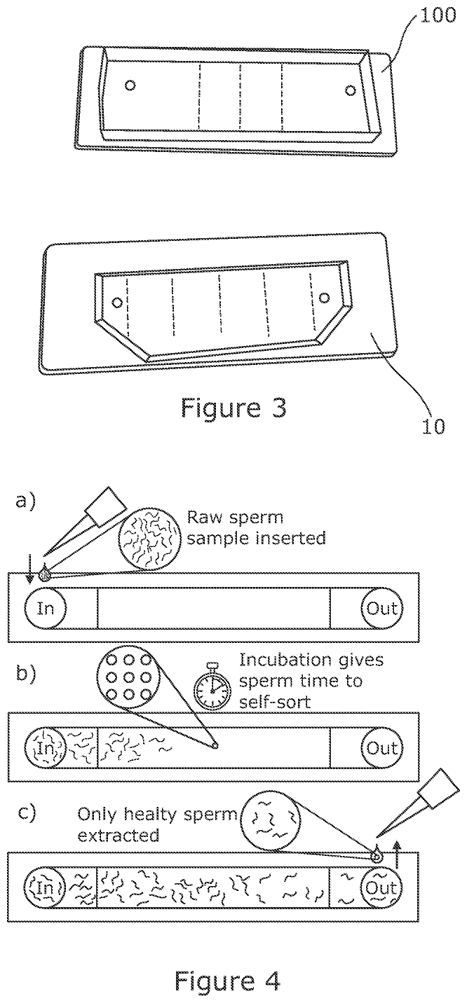

Two microchannel devices were designed, shown in FIGS. 1 and 3. A “non-walled” control device 10 consisted of a single, open channel with surfaces only at the channel edges, whilst the “walled” device 100 discussed above comprised an area subdivided into several channels (width of 200 μm×length of 4 cm), beginning and ending 0.5 cm from loading wells at the inlets 102. 4 cm was the chosen length as a compromise between the distance of in vivo sperm migration and the yields that would be obtained. These two device designs are referred to as “first-generation microchips”.

The channels were cast from polydimethylsiloxane (PDMS) and subsequently fused to a solid PDMS layer above and a glass substrate below, as shown in FIG. 2. Markings were made on the underside of the two device designs prior to use (see FIG. 3) so that measures could be recorded at consistent distance intervals. Non-walled microchip markings were made at every centimetre from 0 cm to 4 cm from the inlet loading well, whilst walled microchip markings were made along the walled region at 1 cm, 2 cm, and 3 cm. The beginning and end of the channels indicated 0 cm and 4 cm in the walled microchips. The extraction zones were defined as the region after the end of the walls in walled microchips, and the region following the 4 cm interval in non-walled microchips.

### Second-Generation Microchannels Microchip Designs

Initial observations suggested that because the channel ends in the walled microchips were curved, sperm cells were able to follow these curves and progress back along the channel away from the extraction zone, reducing the cell yields collected. Furthermore, cells that did leave the channel surfaces could hit the extraction zone end surface, also curved, and similarly moved away from the extraction zone.

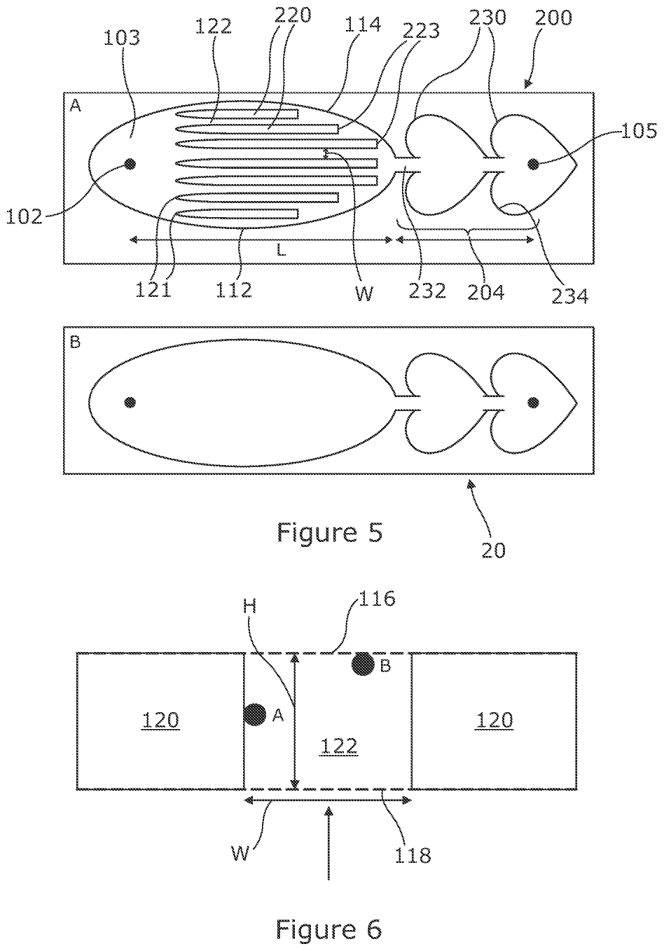

A second-generation device 200 was subsequently designed to mitigate these effects, together with a second-generation control device 20. Both of these microchips are illustrated in FIG. 5.

The second-generation devices share many features with the first generation devices discussed above with respect to FIG. 1. Like reference numerals have been used to indicate like parts, and it should be understood that the features discussed above with respect to the device 100 are equally applicable in the device 200.

The second-generation device 200 includes internal walls 220 that are similar to the internal walls 120 of the first-generation device 100, in that both internal walls 120 and internal walls 220 include a first end 121 (particularly, the end closest to the inlet) that is shaped to encourage sperm cell progression into a sub-channel defined at least in part by that internal wall. In particular, the first end 121 of each of the one or more internal walls is rounded or tapered.

The first-generation device 100 has internal walls with a second end 123 (i.e. closest to the extraction zone) that is similar in shape to the first end. In contrast, a second end 223 of at least one of the one or more internal walls 220 in the second-generation device 200 is shaped to discourage sperm cell progression into a sub-channel 122 defined at least in part by that internal wall. In particular, each of the internal walls 222 comprises a flat surface at an angle to (in particular, perpendicular to) the preferred direction of cell motion.

One or both of the side walls 112, 114 curves or tapers towards the extraction zone 204, such that sperm are directed to the extraction zone as they leave the sub-channels. The second end 223 of each internal wall 220 is located such that the distance a sperm swims freely (without walls) to a side wall is minimized. In the particular example shown the second ends are staggered, and the internal walls have different lengths. In particular, the internal walls closest to the sidewalls are shorter than the interior internal walls. This means the distance from the end of a sub-channel to a curved side wall is similar regardless of the sub-channel location within the channel 106.

The second-generation device 200 is further modified as compared with the first generation device, in that an extraction zone 204 of the device 200 comprises one or more non-return features 230. The extraction zone comprises an entry channel 232, and the non-return features comprise one or more surfaces 234 shaped (for example, curved) to deflect cells away from the entry channel.

FIG. 5 shows a schematic of the second-generation microchip designs, which were constructed from PDMS on a glass substrate in the same manner as the first-generation microchips discussed above. Picture A shows device 200 with a main central void divided into smaller channels (width of 200 μm) each providing a wall surface for sperm to contact and each with a flat end, and picture B shows a control device 20 with the main void not sub-divided into smaller channels. Both control and walled designs have ‘heart’-shaped areas in the extraction zone to restrict backwards cell movement. The path length of both channel designs is 3 cm, and both have equal internal volumes.

### Preparation

To test the efficacy of the new walled device designs as compared with the control (unwalled) designs, the devices were loaded with a suitable cell transit medium. In the examples discussed herein, methylcellulose (4000 CP) (Sigma Aldrich) medium was used to fill the microchips in two concentrations, consisting of 0.5 wt. % and 1 wt. % methylcellulose in modified Earle's Balanced Salt Solution (EBSS). Methylcellulose solutions were mixed for >3 days with constant agitation at 4° C. The medium was added to the chip using a syringe, bubbles were flushed out using an excess of methylcellulose solution, and a small excess volume was left above the loading areas so that air bubbles would not be introduced when adding the loading wells. Three drops of methylcellulose were added to each loading well (inlet and outlet) and the system was incubated overnight at 37° C. and 6% CO₂ to allow the system to equilibrate. Other clinically safe methods of increasing solution viscosity can be used with the device for example hyaluronic acid or polyvinylpyrrolidone. The concentration percentages of these solutions can be varied for the required application.

### Sperm Selection Process

0.5 ml of semen was loaded into the inlet loading well and 0.5 ml of methylcellulose was concurrently added to the outlet well. Microchips were immediately transferred to view under an Olympus IX81 inverted fluorescence microscope using a Photometrics Evolve camera and OptoMorph software. Microchips were viewed live using positive phase contrast microscopy at an objective lens magnification of 10×. The time for the first cell to appear at 0.3 cm from the inlet after sample loading was recorded, along with counts at each marked distance interval along the chip after 30 minutes using the protocol described below. After these 30-minute counts, microchips were returned to the incubator. 30-minute counts were low in 1% methylcellulose, so counts were also performed with the same methods after 60 minutes for all subsequent samples in both methylcellulose concentrations. After two hours, both loading wells were removed for easier visualisation of the extraction zones, and cells in the extraction zones were recorded as the microchip cell yield. One walled and non-walled microchip was run for each sample to give paired experiments between designs. Samples were loaded 15 minutes apart and the design loaded first was alternated to limit the effect of sample incubation time.

### Motility Classification

To accurately record sperm motility behaviours, 61 images across a 30 second period were taken to generate a time-lapse. From these images, cells were recorded as interacting with walls or not, and their direction of travel. Cells meeting walls were defined as those which touched any surface at any point throughout the time-lapse. The recorded direction of cell movement was based on cell position at the final image of the time-lapse compared to its position in the first image. If the final position was closer to the extraction zone than the start position, no matter the path taken, the cell was recorded as swimming forwards. If the end position was closer to the inlet loading well, the cell was recorded as swimming backwards. If the cell moved off-screen during the time-lapse then the direction it was swimming whilst it was visible was recorded. The proportion of cells falling into each category was calculated for each microchannel system.

### Cell Concentrations

To accurately count cells at various distances, time-lapses were generated from 21 images across a 10 second period, which was later increased to 51 across 10 seconds to increase clarity. Five time-lapses were taken at each distance interval along the chip. For consistency, both edges of the microchip were included, and three further time-lapses were taken from the centre of the chip to get a fully representative value. Similar rules were followed in the extraction zone for the final counts after 120 minutes. All cells with a flagellar beat were counted and cells per objective were recorded.

Combining the known depths of the microchips, 36 μm for first-generation chips and 28 μm in second-generation chips, with the objective area, calculated using the haemocytometer, meant that cell concentrations could be calculated from the counts. Due to the channels in the walled chips, only 50% of the objective area was available for cells to swim through at 1 cm to 3 cm and 75% was available at 0 cm and 4 cm. This effect was accounted for when calculating the cell concentrations. Concentrations at the final distance interval were also calculated as a percentage of the concentration present at the first distance interval as a measure of proportional cell progression through the chips. For a suitable comparison of this measure between generations, it was also calculated across the same distance in equivalent chip designs between the two generations. It is noted that concentrations taken from these time-lapses produced higher concentrations than if instantaneous counts were performed. Cell motility behaviours were also recorded according to the above protocol from these images.

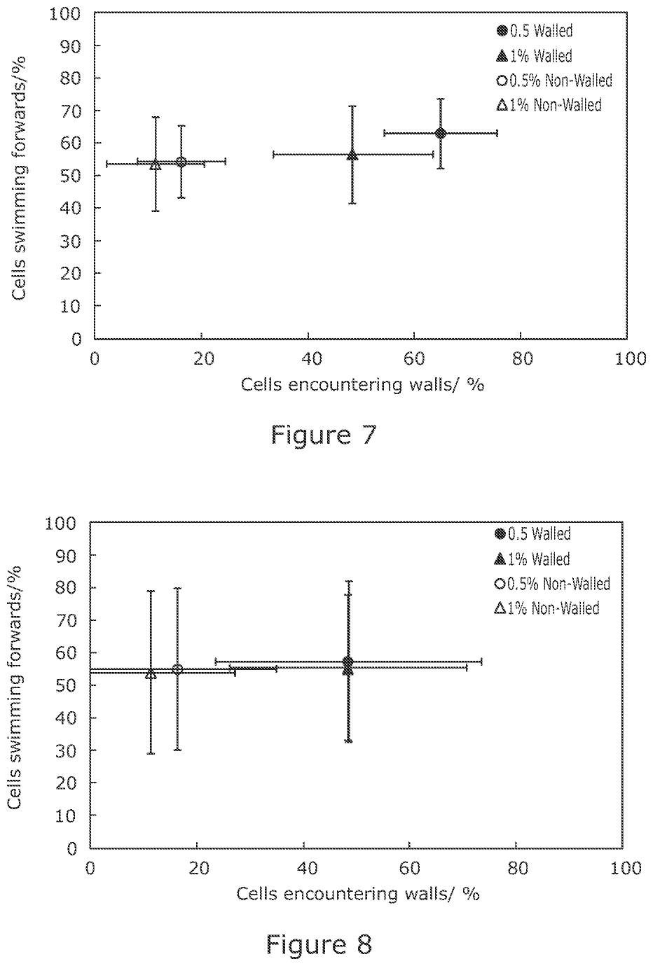

FIG. 7 shows the proportion of cells meeting walls in each first-generation system plotted against the proportion of cells swimming forwards. Both chip designs filled with 0.5% methylcellulose had 20 replicates performed. 1% methylcellulose walled and non-walled chips had 11 and 12 replicates performed respectively. Error bars are standard error. Walled chips had a greater proportion of cells meeting walls (p<0.01) than in corresponding non-walled chips regardless of methylcellulose concentration, but only walled chips filled with 0.5% methylcellulose had more cells swimming forwards (p=0.27). Walled chips filled with 0.5% methylcellulose had a slight increase in the proportion of cells swimming forwards than those filled with 1% (p=0.47).

FIG. 8 shows similar data for the second-generation chips, and shows the proportion of cells meeting walls in each second-generation system plotted against the proportion of cells swimming forwards. Error bars are standard error. Four replicates were performed for both chips filled with 0.5% methylcellulose, and 1% methylcellulose walled and non-walled chip values had six and five replicates performed respectively. Walled chips saw more cells meeting walls than in non-walled designs irrespective of media (p<0.06), but the direction of motility was similar (p>0.88). Methylcellulose concentration had no effect on these trends (p>0.75).

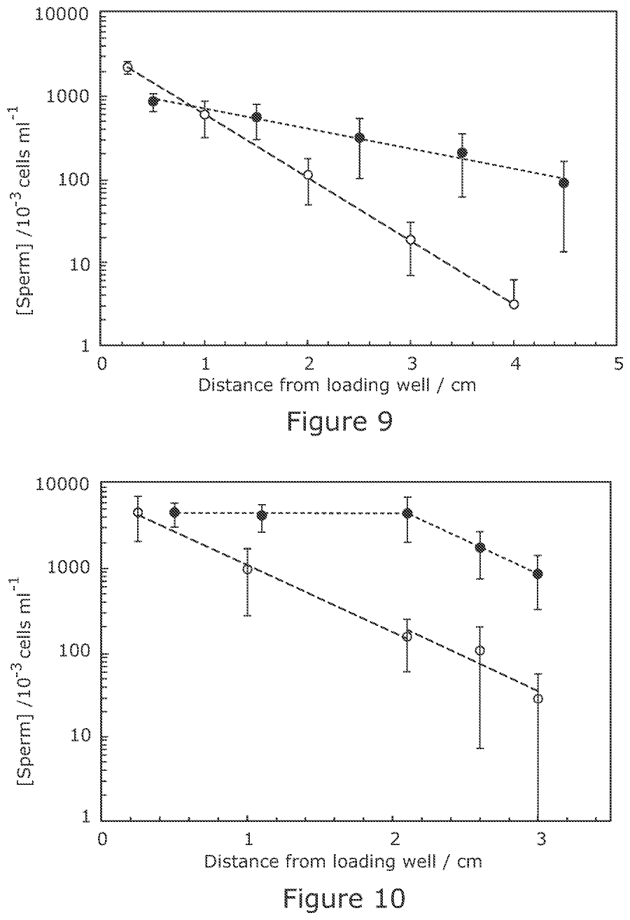

FIG. 9 shows average cell concentrations at 1 cm intervals along the first-generation microchannel chips 30 minutes after loading the sample. Values are normalised by concentrations of progressively motile cells in the corresponding sample and are plotted on log scales. Error bars are standard error. Percentage of cells reaching the end was found to be 3.9 for walled devices and 0.1 for the non-walled (p=0.1284).

FIG. 10 shows similar data for the second-generation microchips. Percentage of cells reaching the end was found to be 13.8 for walled devices and 0.3 for the non-walled (p=0.1388).

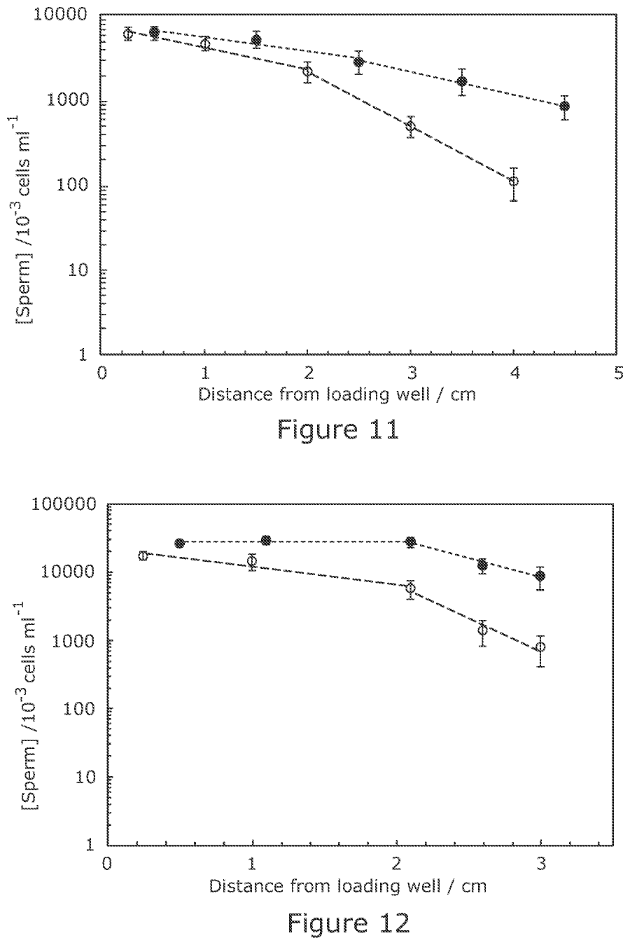

FIG. 11 shows average cell concentrations at 1 cm intervals along the first-generation microchannel chips 60 minutes after loading the sample. Values are normalised by concentrations of progressively motile cells in the corresponding sample and are plotted on log scales. Error bars are standard error. Percentage of cells reaching the end was found to be 13.0 for walled devices and 1.6 for the non-walled (p=0.0009).

FIG. 12 shows similar data for the second-generation microchips. Percentage of cells reaching the end was found to be 34.8 for walled devices and 4.2 for the non-walled (p=0.0524).

These results suggest that the microchannel designs described herein, utilising internal walls between the inlet and extraction zone, are more effective at separating motile sperm from non-motile sperm than non-walled designs which have an otherwise similar shape and structure. This is believed to be because these walled devices mimic much more the in-vivo situation by providing the cells a multitude of surfaces to progress along, analogous to migration along the walls of the cervix and the fibrils in cervical mucus. Motile sperm cells align to the walls, greatly reducing their random walk behaviour and enabling them to rapidly move along the channel towards the extraction zone. Having a multitude of walls in a channel effectively supplies a massive surface for them to migrate along increasing the available “zone for optimal migration”; thereby also reducing likelihood of cell collision as they progress.

The surface area to volume ratio within the channel 106 is substantially increased in the walled devices as compared with the non-walled devices. This provides increased concentration of motile cells at shorter preparation times. The surfaces area to volume ratio of a channel including orienting features, such as walls, may be more than 20% greater than the surface area to volume ratio of a channel not including orienting features, for example 30%, 40%, 50% or 60% greater.

It will be appreciated that the walls described herein are only examples of possible orienting features, and that other wall shapes may be possible to those depicted in the Figures.

It will be appreciated by one skilled in the art that changes may be made to the examples described above within the scope of the claims set out below. Features from different examples are combinable together. It is thus to be understood that the invention is not limited to the examples described above but is instead defined by the scope of the claims.

1. A microchannel device for preparation of sperm, the device comprising:
an inlet;
an extraction zone; and
a channel fluidly connecting the inlet to the extraction zone;
wherein the channel comprises a plurality of orienting features operable to bias a direction of sperm cell movement within the channel towards a preferred direction; and
wherein the channel has a length of approximately 1 centimetre or greater.

2. The microchannel device of claim 1, wherein the channel comprises a first side wall, a second side wall, a top wall and a bottom wall, together defining a channel volume,
wherein the plurality of orienting features are located in the channel so as to increase the surface area to volume ratio of the channel as compared with a channel absent said orienting features.

3. The microchannel device of claim 1 or claim 2, wherein the orienting features comprise one or more internal walls disposed within the channel so as to define a plurality of sub-channels.

4. The microchannel device of claim 3, wherein the device comprises more than 4 internal walls, more than 5 internal walls, between 5 and 20 internal walls, or between 7 and 10 internal walls.

5. The microchannel device of claim 3 or claim 4, wherein each subchannel has a width of between 10 μm and 200 μm.

6. The microchannel device of any one of claims 3-5, wherein a first end of at least one of the one or more internal walls is shaped to encourage sperm cell progression into a sub-channel defined at least in part by that internal wall.

7. The microchannel device of claim 6, wherein the first end of each of the one or more internal walls is rounded or tapered, said first ends being located closest to the inlet.

8. The microchannel device of any one of claims 3-7, wherein a second end of at least one of the one or more internal walls is shaped to discourage sperm cell progression into a sub-channel defined at least in part by that internal wall.

9. The microchannel device of claim 8, wherein the second end of each of the one or more internal walls comprises a flat surface at an angle to (for example, perpendicular to) the preferred direction, said second ends being located closest to the extraction zone.

10. The microchannel device of claim 2 or any preceding claim dependent on claim 2, wherein one or both of the side walls tapers towards the extraction zone, such that sperm are directed to the extraction zone.

11. The microchannel device of claim 2 or any preceding claim dependent on claim 2, wherein the second end of each internal wall is located such that the distance a sperm swims freely (without walls) to a side wall is minimized.

12. The microchannel device of any preceding claim, wherein the extraction zone comprises one or more non-return features.

13. The microchannel device of claim 12, wherein the extraction zone comprises an entry channel, and the non-return features comprise one or more surfaces shaped to deflect cells away from the entry channel.

14. The microchannel device of claim 13, wherein the surfaces are curved.

15. The microchannel device of any preceding claim, further comprising a cell transit medium within the device.

16. The microchannel device of any preceding claim, wherein the channel and orienting features are formed of one or more of polydimethylsiloxane (PDMS), polypropylene, polyethylene, nylon, polyetheretherketone (PEEK), polyethylene terapthalate (PET), polybutylene terephthalate (PBT), EVOH, acrylic, ABS, polycarbonate, polysulfone, polystyrene, polyethersulfone (PES), polyphenylsulfone (PPSU) and polyvinyl chloride (PVC).

17. A microchannel device for preparation of sperm, the device comprising:
an inlet;
an extraction zone; and
a plurality of sub-channels fluidly connecting the inlet to the extraction zone;
wherein each sub-channel has a length of approximately 1 centimetre or greater.

18. The microchannel device of claim 14, further comprising the features of any one of claim 5, 13, 14 or 15.

19. The microchannel device of any preceding claim, further comprising a monitoring region.

20. A method of sperm preparation, the method comprising:
loading a sample comprising sperm cells into an inlet of a microchannel device as set out in any one of claims 1-19;
allowing sperm cells to transit towards the extraction zone; and
extracting sperm cells from the extraction zone of the microchannel device.

21. The method claim 20, comprising allowing sperm cells to transit towards the extraction zone for at least 15 minutes, for at least 30 minutes, for at least 45 minutes, or for at least 60 minutes.

22. The method of claim 20, comprising allowing sperm cells to transit towards the extraction zone for between 15 and 75 minutes, for approximately 30 minutes or for approximately 60 minutes.
